(19) United States
(12) Patent Application Publication
(10) Pub. No.: US 2013/0259814 A1
(43) Pub. Date: Oct. 3, 2013

(54) LONG WEAR COSMETIC COMPOSITIONS CONTAINING POSS THERMOPLASTIC ELASTOMERS

(71) Applicants: Emmanuel Dimotakis, Oradell, NJ (US); Hy Si Bui, Piscataway, NJ (US); Mohamed Kanji, Edison, NJ (US)

(72) Inventors: Emmanuel Dimotakis, Oradell, NJ (US); Hy Si Bui, Piscataway, NJ (US); Mohamed Kanji, Edison, NJ (US)

(73) Assignee: L'OREAL S.A., Paris (FR)

(21) Appl. No.: 13/992,073

(22) PCT Filed: Dec. 7, 2011
(86) PCT No.: PCT/US11/63733

§ 371 Date: Jun. 6, 2013

Related U.S. Application Data
(60) Provisional application No. 61/420,481, filed on Dec. 7, 2010.

(51) Int. Cl. A61K 8/91; A61Q 1/06; A61Q 1/02; A61Q 5/06; A61Q 1/04; A61Q 1/00; A61Q 3/00

(52) U.S. Cl. CPC A61K 8/91; A61Q 1/00; A61Q 1/06; A61Q 3/00; A61Q 5/065; A61Q 1/04; A61Q 1/02
USPC 424/61; 514/772.1; 424/63; 424/64; 424/70.121; 424/70.6

(57) ABSTRACT

Disclosed are cosmetic compositions and methods of making up keratinous tissue in a way that preserves long-wear but also greater comfort, reduced tackiness and enriched color, and which entail use of polyhedral oligomeric silsequioxanes (POSS)-grafted polyolefins.

### CROSS-REFERENCE TO RELATED APPLICATIONS

The present application claims the benefit of the filing date of U.S. Provisional Application No. 61/420,481, filed Dec. 7, 2010, entitled LONG WEAR COSMETIC COMPOSITIONS CONTAINING POSS THERMOPLASTIC ELASTOMERS, the disclosure of which is hereby incorporated herein by reference.

### BACKGROUND OF THE INVENTION

Cosmetic manufacturers continuously explore new avenues in search of cosmetic ingredients and combinations thereof that will fill the always needed gaps of longer wear, richness of color, comfort and non-tackiness. Cosmetic formulations are sought that provide long-lasting, durable wear, preferably one or more days, removable by the consumer whenever desired, and exhibit a rich and natural-looking color. In response to this need, the cosmetics art has developed numerous extended wear technologies, among them formulations that contain a combination of solid pigments, silicone resins such as organosiloxanes and diorganopolysiloxanes. Although these types of formulations have extended wear performance, they have also been found to be tacky or sticky during or after product application, and even cause unpleasant sensations of tautness, making these cosmetics uncomfortable to wear. It has also been found that a film formed on the skin after application is too rigid also causing unpleasant sensations such as, for example, during facial movements. Beyond issues of tackiness and comfort, silicone-based cosmetic formulations have also been limited from the standpoint of providing a relatively long-lasting rich and natural-looking color to keratinous tissue. Organic pigments are desirable in this respect because they provide a very rich intensity that inorganic pigments tend to lack. However, since most organic colorants are water-soluble, it is difficult to incorporate them into long-wearing cosmetics which is further complicated when the user comes into contact with water from perspiration, raindrops, etc. Use of organic pigments is also compromised from the standpoint that since they are not compatible in non-aqueous systems at appreciable concentrations, they cannot be used in amounts large enough to impart significant color to the composition.

Therefore, there is a remaining need in the cosmetics art to provide long-wearing cosmetic compositions based on film-forming silicone resins and which also provide non-tackiness, greater comfort and a long-lasting color effect.

### BRIEF SUMMARY OF THE INVENTION

The present invention provides novel cosmetic compositions and methods of making up keratinous tissue in a way that preserves long-wear but also greater comfort, reduced tackiness and enriched color. The present invention exploits the use of polyhedral oligomeric silsequioxanes (POSS)-grafted polyolefins.

Accordingly, a first aspect of the present invention is directed to a cosmetic composition that contains a polymer having a chain that contains a polyolefin, and a polyhedral oligomeric silsesquioxane (POSS) grafted onto the polymer chain, a solvent and at least one other cosmetically acceptable ingredient. In some embodiments, wherein monomeric POSS (the POSS molecule that is reacted with the polyolefin) includes a functional group reactive with a vinyl group on the polyolefin, and the POSS is grafted onto the polymer chain via the functional group. In other embodiments, monomeric POSS includes a polymerizable functional group, and the polymer chain further includes the polymerized functional group such that the POSS is grafted onto the polymer chain via the polymerized functional group (the polymerizable functional moiety becomes integrated into the polymer backbone). In other embodiments, more than one polyolefin component is present. Thus, the polymers of the present invention include random and non-random copolymers, and block copolymers and terpolymers.

A second aspect of the present invention is directed to a method for making up keratinous tissue, which entails applying to the keratinous tissue (e.g., skin, lips, eyelids, hair, nails) a cosmetic composition that contains a polymer having a chain that contains a polyolefin, and a polyhedral oligomeric silsesquioxane (POSS) grafted onto the polymer chain, a solvent and at least one other cosmetically acceptable ingredient.

A third aspect of the present invention is directed to a method for making a cosmetic composition, which includes formulating a composition containing a polymer having a chain that includes a polyolefin, and a POSSPOSS grafted onto the polymer chain, a solvent and at least one other cosmetically acceptable ingredient, into a cosmetic composition.

The POSS component provides the benefits of long wear but without compromising with respect to comfort, tackiness and color enrichment. The polyolefin component of the polymer has a molecular weight of about 1,000 to about 200,000 daltons, and a relatively low glass transition temperature (Tg) (e.g., less than about 25 C.). Without intending to be bound by any particular theory of operation, Applicants believe that backbone of the relatively low Tg polyolefin provides adequate tackiness and adhesive properties that causes aggregation, resulting in excellent comfort and adhesion both to skin and colorants, but without a prolonged tacky sensation.

### DETAILED DESCRIPTION

Other than in the operating examples, or where otherwise indicated, all numbers expressing quantities of ingredients and/or reaction conditions are to be understood as being modified in all instances by the term “about,” meaning within 10% to 15% of the indicated number.

“Keratinous tissue”, as used herein, includes but is not limited to, skin, hair and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen-containing groups, ester groups, thiol groups, sulphonate groups, thiosulphate groups, siloxane groups, and polysiloxane groups. The substituent(s) may be further substituted.

“Volatile”, as used herein, means having a boiling point of less than about 100° C. “Non-volatile”, as used herein, means having a flash point of greater than about 100° C.

As used herein, the terms “at least one”, “a”, and “an” mean one or more and thus include individual components as well as mixtures/combinations.

“Long wear” compositions as used herein, refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Hardness” as used herein, refers to the resistance of a composition to penetration. Hardness may be evaluated according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed.

### The POSS-Grafted Polyolefins

Polyolefin Component:

The polyolefin component of the polymers of the present invention (and in the case of block copolymers, the total polyolefin component) has a molecular weight of from about 1,000 to about 200,000 daltons. In general, the polyolefin component has a glass transition temperature (Tg) less than about 25° C. The term “glass transition temperature” generally refers to the temperature at which the amorphous material changes from a glassy solid state to a rubbery state. This temperature may be measured by standard techniques in the art, such as DSC (Differential Scanning calorimetry), e.g., according to the ASTM D3418-97 standard. As used herein, the term “about” as it is used in the specific context of Tg allows for imprecision in the use of a particular technique, or the variation between or among various techniques, in determining Tg. Thus, the term provides variability in the order of ±2° C. Polyolefin/POSS polymers wherein the polyolefin has a Tg in this range contribute to desirable cohesive properties, and particularly to particulate colorants, producing colored cosmetic compositions that exhibit excellent wear. This property of the polyolefin component also results in a polymer that exhibits excellent aggregation characteristics, producing cosmetic compositions that exhibit excellent tackiness and comfort.

The polyolefins for use in the present invention are non-silicone based polymers. They have the values of elastic or storage modulus G′, at frequency of 1 Hz and 25° C., as measured by Dynamic Mechanical Analyzer (DMA), that generally ranges from about 1 Mpa to about 100 Mpa, or higher. See, “An introduction to rheology” by H. A. Barnes, J. F. Hutton and K. Walters, pages 46 to 54 (published by Elsevier 1989). The polyolefins have a refractive index (RI) which, in general, is greater than 1.46. Representative examples of polyolefins that may be present in the polymers of the present invention include polyethylene, polypropylene, polyisobutene, polybutene, polyisoprene, polybutadiene, polycycloalkenes (e.g., polycyclooctene) and poly-hydrocarbon-dienes [e.g., pentylene-dienes, and olefinic copolymers thereof]. More than one polyolefin component may be present (e.g., polyethylene/polypropylene, styrene/butadiene/styrene, polyethylene/polypropylene/styrene and polycyclooctene/styrene).

The POSS Component:

The other component of the polymers of the present invention is a polyhedral oligomeric silsesquioxane (POSS), which is a general name to describe organic-inorganic materials with a cubic caged (“complete” or “incomplete”) core structure that contains silicon and oxygen atoms, with a silicon to-oxygen ratio of 1 to 1.5. See, Johshi, et al., J. Macromolecular Sci, Part C: Polym. Rev. 2004, 44, 389; Wang, et al., J. Inorg. Organomet. Polym. 2001, 11, 123. The POSS core is surrounded by peripheral groups off each silicon atom (also referred to as a POSS periphery), which can consist of a wide variety of non-reactive and reactive functional groups alike, including for example, aliphatic, aromatic, or aryl groups, as well as any other functional groups provided that the derivatized POSS is not rendered unacceptable for cosmetic purposes.

In some embodiments, the POSS molecules have the complete cage structure of Formula I formula III which have 8 and 6 silicon atoms respectively, as follows:

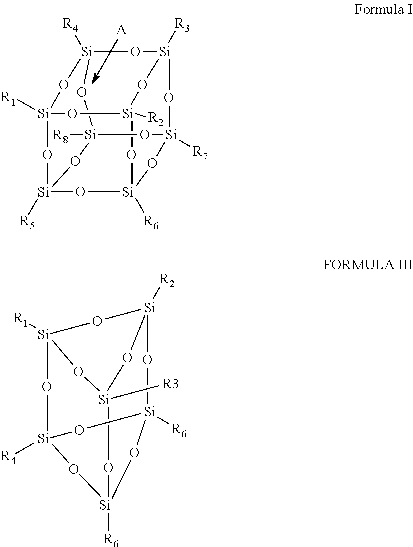

In other embodiments, one or even two of the oxygen bridges between successive silicon atoms are broken or missing, in which case the “POSS” is referred to as having an “incomplete” case structure. Representative examples include the three-dimensional cage structures illustrated in Formulas IIA-E, IVA-E and V, as follows:

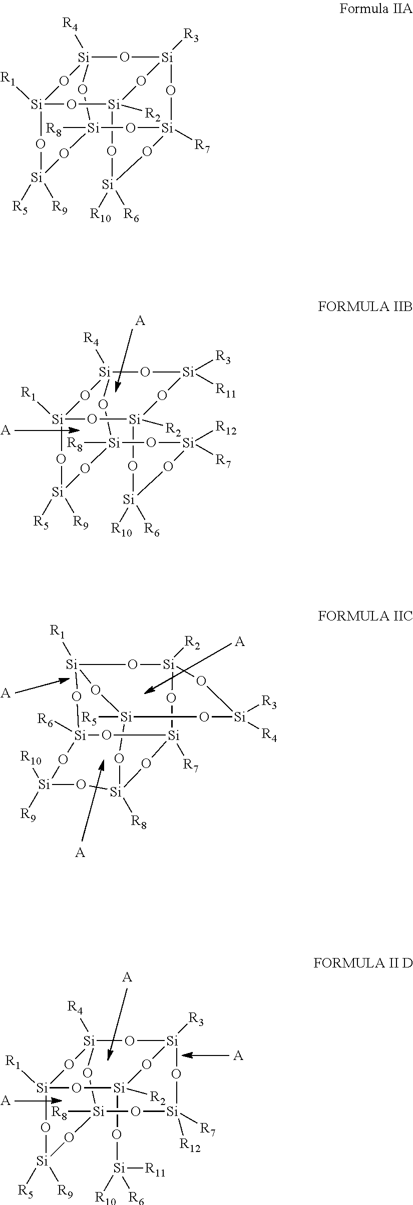

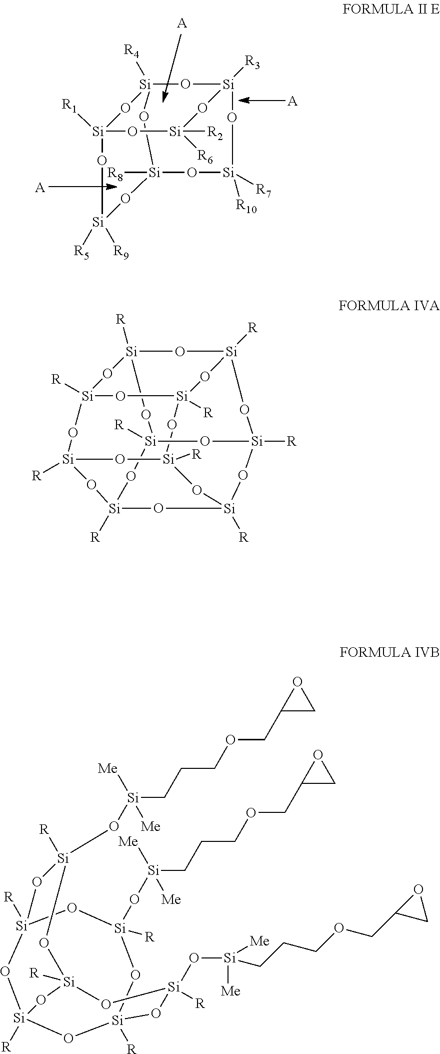

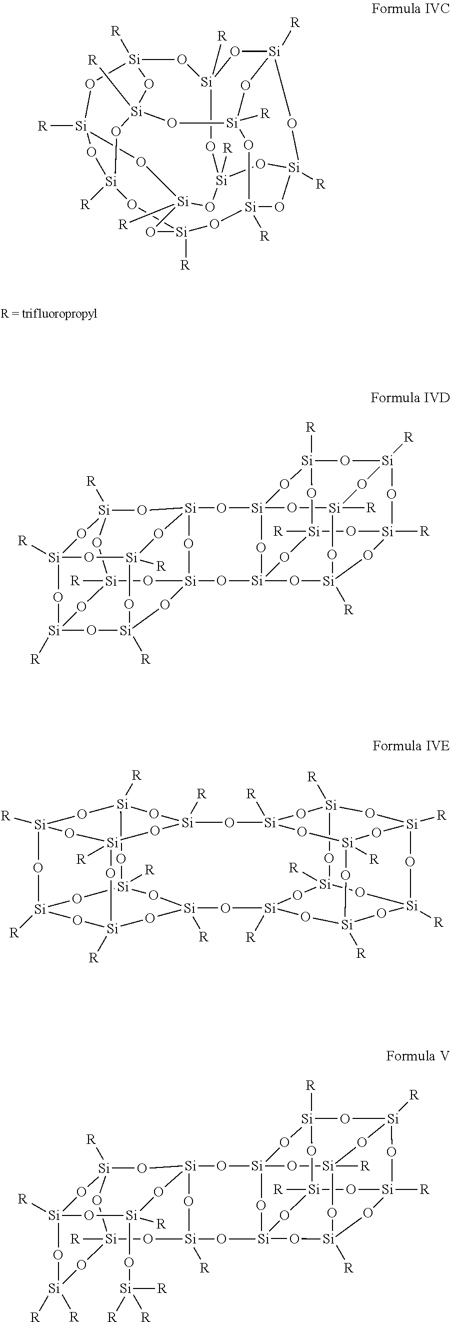

In Formula IVA, the number of Si atoms in the cage is 10, in Formula IVB, the number of Si atoms is 10 and in Formula IVC, the number of Si atoms in the cage is 12. In Formulae IVD and IVE, the number of Si atoms in the cage or core is 16. In the “incomplete” cage structure shown in Formula V, one or more of the oxygen bridges between successive silicon atoms is broken or missing. Even though Formula IVC is shown with specific R groups, the structure is not limited to that exact molecule—the R groups may include other substituents as disclosed hereinbelow.

To make the POSS-grafted polyolefins of the present invention, the POSS periphery must contain a functional group reactive with pendant vinyl groups on various polyolefin backbones, and/or a functional group that is itself polymerizable and can become integrated into the polyolefin backbone. These reactive groups may be linked directly or indirectly to the Si atom in the POSS (e.g., the reactive group may be attached to an “R” group). Thus, the polymers of the present invention include POSS grafted onto homo-polyolefins, and polyolefin copolymers including polyolefin block copolymers and terpolymers that contain, in addition to the polyolefin component(s), blocks of the polymerized functional group. For purposes of the present invention, both types of embodiments are embraced by the term “grafted”. These groups are described in the context of the immediately succeeding section on methods of making the polymers. Other substituents that may be included in the POSS periphery are discussed thereafter. The POSS-grafted polyolefins may be cross-linked or non-crosslinked.

### Methods of Making the POSS-Grafted Polyolefins

The polyolefins grafted with POSS may be synthesized in accordance with a variety of synthetic techniques known in the art. See, generally, Wu, et al., J. Macromolec. Sci. Part C: Polymer Reviews 19:25-63 (2009). For example, the POSS-polyolefin polymers may be in the form of random or non-random copolymers, diblock and triblock copolymers and tadpole-shaped copolymers.

In some embodiments, the POSS is derivatized with a polymerizable moiety that is capable of being integrated into the polyolefin backbone. Suitable polymerizable moieties include ethylenically unsaturated groups (e.g., alkenyl groups and preferably vinyl groups) and epoxy groups. Ethylenically unsaturated groups, especially those that can be polymerized by means of a free radical mechanism e.g., substituted and unsubstituted acrylates, methacrylates, alkenes and acrylamides, are preferred. Norbornene and styrene are two such examples of such polymerizable moieties. Other examples include functional silicones—for example, silanes (Si—H), and silanols, hydroxy, urethane, acrylate, vinyl, amides, MQ or T groups, functional acrylates, functional polyamides, PVK, PVA, PS, PEG, PPG, polysaccharides or modified starch, functional block copolymers, functional polyesters and polyesters, fluorinated polymers and wax. Persons skilled in the art would be able to select yet other polymerizable moieties e.g., from among the POSS substituents disclosed herein, and in the scientific literature. Thus, in these embodiments, the monomeric derivatized POSS and the olefin(s) are reacted together to form olefin-POSS copolymers (and in the case of two olefins, terpolymers). See, Seurer, et al., Macromol. Chem. Phys. 209:1198-1209 (2008), which describes a synthetic procedure in which POSS, linked to a diene such as norbornene, via a POSS periphery containing ethyl, isobutyl or phenyl groups, was polymerized with ethylene and propylene, thus making ethylene/propylene/POSS terpolymers via ring-opening metathesis polymerization (ROMP), and using the polymerization catalyst ethyl(bis-indenyl)hafnium dichloride.

In other embodiments, POSS can be grafted onto polyolefin backbones that as a result of the polymerization of the olefin (such as isoprene and butadiene), have pendant (also known as “dangling”) vinyl groups (e.g., 1,2-butadienes). In these embodiments, the POSS periphery is derivatized with a functional group reactive with the vinyl group on the polyolefin backbone. This type of polymerization can be practiced using a Zieglar-Natta scheme (see, e.g., Bhowmich, et al, Handbook of Elastomers, 2^{nd} Ed., Marcel Dekker, Inc., New York 2001) using polymerization initiators including for example, benzoyl peroxide, azobisisobutyronitrile (AIBN), the initiators available from Akzo Nobel under the tradenames Trigonox® and Perkadox® e.g., Trigonox 21S (tert-butyl peroxy-2-ethylhexanoate), Trigonox 25C75 (tert-Butyl peroxypivalate), Trigonox 141 (2,5-dimethyl-2,5-di(2-ethylhexanoylperoxy)hexane), and Perkadox 16 (di(4-tert-butylcyclohexyl) peroxydicarbonate). See, for example, Chun, et al., Mat. Res. Soc. Symp. Proc. 661:K.K.10.8.1-K.K.10.8.6 (2001), which reports dimethyl silane-POSS grafted onto a polyisoprene-polystyrene block copolymer. See, also, Fu, et al., Macromolecules 37:5211-18 (2004); Drazkowski, et al., Macromolecules 39:1854-63 (2006); and Drazkowski, et al., Macromolecules 40:2798-2805 (2007), which report grafting of styrene-butadiene-styrene triblock copolymers grafted with the following octameric POSS ([Si₉O₁₂]) derivatives, each containing a single silane functional group (i.e., ((CH₂)₃SiMe₂(C₆H₄)(SiMe₂H)) and an “R” group, as follows: cyclopentyl (i.e., (c-C₅H₉); cyclohexyl; cyclohexenyl; and phenyl; and isobutyl, respectively.

Even further, Zheng, et al., J. Poly. Sci. Part A: Polymer Chemistry 39:2920-28 (2001), report a two-step synthetic route for the preparation of PE-POSS copolymers, via ROMP of cyclooctene and a POSS having a periphery containing a single norbornene group and 7 cyclopentyl groups (also referred to as cyclopentyl-POSS-norbornene macromonomer 1-[−(5-norbornen-2-yl)ethyl]-3,5,7,9,11,13,15-heptacyclopentylpentacylclo[9.5.1.1.1.1]octasiloxane), using Grubb's catalyst, wherein a large degree of control over incorporation of the POSS monomer was achieved by use of diimide reduction which completely removed unsaturated units from the polymer backbone. In Zheng, et al., Macromolecules 34(23):8034-39 (2001), control over the incorporation of cyclopentyl-POSS-norbornene macaromonomer with ethylene and also with propylene using classical metallocene catalysts. In Zheng, et al., Macromolecules 37(23):8606-11 (2004), polymerization of cyclopentyl-POSS-norbornene macaromonomer with polybutadiene using Grubb's catalyst, wherein the resultant polymers formed two-dimensional lamellar-like nanostructures of assembled cubic silsesquioxanes, is reported.

Synthesis of tadpole-shaped (monochelic) POSS-containing hybrid (alkyne-terminated) polystyrene via CuBr-catalyzed click coupling is reported in Zhang, et al., Polymer 51(10):2133-39 (2010).

Persons skilled in the art would be able to select yet other functional groups reactive with vinyl groups e.g., from among the POSS substituents disclosed herein, and in the scientific literature.

In some embodiments, the polyolefin component is a block copolymer containing units of polyethylene and polypropylene, and the POSS component contains 8, 10 or 12 Si atoms, wherein one of the “R” groups is the reactive group or the polymerizable functional group, and all other R substituents are isobutyl or a cycloalkyl or cycloalkenyl (or wherein the reactive group or the polymerizable functional group is directly attached to an R substituent which thus serves as a linker).

### Other POSS Substituents

In addition to the functional group reactive with a vinyl group and/or a polymerizable functional group, the POSS may have any substituent (including non-reactive and additional reactive groups) as described hereinbelow. For example, the POSS may have at least one M, D or T subunit.

The “M” unit can be represented by the structure:

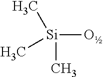

The “D” subunit can be represented as:

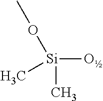

The symbol “T” denotes the trifunctional subunit, (CH₃)SiO_{3/2} and can be represented as:

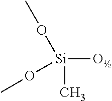

Preferably, at least four of the Si atoms in the POSS structure are “completely saturated.” Most preferably, all of the Si atoms are bound, through oxygen atoms, to three other Si atoms within the cage as shown in Formulas I, III and IVA, thus all the Si atoms are “completely saturated.” While illustrated in Formula I as Si atoms, the groups at each corner may be the same or different and may be one or more atoms or groups including, without limitation, silicon, silane, siloxane, silicone or organometallic groups.

Any methyl group can be replaced in the “M”, “D” and/or “T” subunits with another functional or R group. As non-limiting examples, one or more methyl groups could be replaced with another alkyl group, alkene, alkyne, hydroxyl, thiol, ester, acid, ether. In one embodiment, the “IR groups” of the present invention include, without limitation, one or more of the following: methyl, ethyl, propyl, isobutyl, isooctyl, phenyl, cyclohexyl, cyclopentyl, —OSi(CH₃)₂—CH₂—CH₂—(CF₂)₅CF₃, —(CH₂)₃SH, N⁺(CH₃)₃, O—N⁺CH₃)₃, —OH, —(CH₂)ₙN⁺H₃X— wherein n is 0-30 and X is a counter ion,

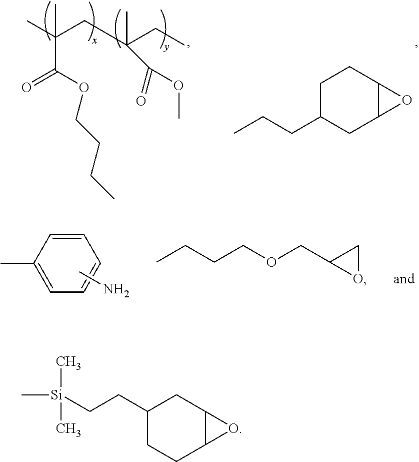

Preferably, the R group is an isooctyl group. These substituent groups may be bound directly to the cage structure or may be bound through a bridging molecule such as an azo, diazo, epoxy or halogen containing material.

For example, the one remaining bond of each silicon of Formula I, III and IVA can bind to a variety of substituents or groups specified, as “R” groups (R₁-R₈), ((R₁-R₆) in Formula III). In some embodiments illustrated in Formulas II, IVB and V a POSS molecule in which one or two of the oxygen bridges between adjacent silicon molecules have been eliminated, a greater number of R groups are possible. When a POSS having 8 Si atoms is employed, it is preferred that no more than two of these inter-silicon connections (oxygen bridges) be eliminated. However, it is possible to eliminate as many as three such bridges (Formula IIE). More preferably, only a single oxygen bridge would be eliminated (Formula IIA). As stated above, the Si molecules not completely bound may have one or more additional positions available for binding additional substituents. In the case of a single missing side, the POSS molecule may include additional R group R₉ and R₁₀, which may be the same or different as the R group R₁-R₈. When 2 or 3 bridges are missing, the POSS molecule may include additional R groups R₉, R₁₀, R¹¹ and R₁₂ (as appropriate), which all may be the same or different and may be the same as the groups identified for R₁-R₈.

In general, R groups (for example, R₁, R₂, R₃, R₄, R₅, R₆, R₇, R₈, R₉, R₁₀, R¹¹ and R₁₂ as shown in the figures and any other R groups appropriate) can be the same or different and may be reactive or nonreactive groups. They may be, in replacing a methyl or H, for example, hydroxy (—OH), alkane derivatives (missing a hydrogen) also known as alkyl groups (other than methyl), alkenyl groups also referred to as derivatives of alkenes (having one or more double bonds), usually missing an H where they are bound to Si in POSS or to some other molecule, alkynyl groups also referred to as derivatives of alkynes (having one or more triple bonds) usually missing an H where they are bound to Si in POSS or to some other molecule, aryl groups (either the 6-carbon ring of benzene or the condensed 6-carbon rings of other aromatic derivatives such as naphthalene) also referred to as derivatives of arenes, usually missing an H where they are bound to Si in POSS or to some other molecule, acyl groups (organic acids without the OH group, e.g., CH₃CO— or C₆H₅CO—), alkoxy groups (alkyl radicals attached to the remainder of a molecule by oxygen), such as methoxy, ester groups, acid groups, acrylate groups, alkyl acrylate groups, hydroxy groups, halogens, amino groups, alkylamino groups, aminoalkyl groups, groups containing one or more tertiary or quaternary nitrogens, silicone containing groups, sulfur containing groups, epoxides, azo groups, diazo groups, halogens, cyclic compounds which can undergo ring opening polymerization or ring opening metathesis polymerization. R groups may also be monomers or polymers where POSS will be used as a pendant substituent of the polymer. Acrylates and cationic polymers providing conditioning properties are provided in one embodiment.

Where appropriate, any of these R groups may themselves be substituted or unsubstituted, saturated or unsaturated, linear or branched. Possible substitutions include C₁-C₃₀ alkyl groups, C₁-C₃₀ alkenyl groups, C₁-C₃₀ alkynyl groups, C₆-C₁₈ aryl groups, acyl groups, alkoxy groups, carboxy groups, ester groups, acrylate groups, alkyl acrylate groups, trihydroxy groups, amino groups, alkylamino groups including mono and dialkylamino groups, mono and dihydroxy alkylamino groups, cyano groups, aminoalkyl groups, groups containing one or more tertiary or quaternary nitrogens, silicone containing groups, sulfur and/or phosphorous containing groups, SO₂X, SO₂X, where X is H, methyl or ethyl, epoxides, azo groups, diazo groups, halogens, cyclic compounds which can undergo ring opening polymerization or ring opening metathesis polymerization (ROMP). Indeed, any group which can be attached to a corner of a POSS molecule can be used.

When these R groups are carbon containing fatty acids or fatty alcohols, aromatic or cyclic groups, they generally may contain between six and 50 carbon atoms and may be saturated or unsaturated, substituted as discussed above or unsubstituted and branched or linear, as appropriate for a given group.

More specifically, possible R groups include, without limitation, hydroxy groups including mono or poly hydroxy groups, phenols, alkoxy, hydroxy alkyls, silanes, amino and in particular, quats, halosilanes, epoxides, alkyl carbonyls, alkanes, haloalkyls, halogens, acrylates, methacrylates, thiols, nitriles, norbornenyls, branched alkyl groups, polymers, silanes, silanols, styryls and thiols. In a single POSS molecule of Formula I, R₁ could be H, R₂—OH, R₃—NH₂, R₄—CH₂CH₂N⁺CH₃ (OCH₂CH₃) CH₂CH₂CH₃, R₅—CH₂CH₂CHOCH₂ (epoxide), R₆—OC(CH₃)₃, R₇—OOC(CH₂)₁₆CH₂ and R₈ could be Cl. This is a hypothetical example, merely to illustrate that each of the R groups can be derivatized separately and to emphasize the wide variety of possible substitutions.

In one embodiment, these POSS molecules are not completely substituted with the same R groups (e.g., not all R₁-R₆, R₁-R₈, R₁-R₁₀ or R₁-R₁₂ (and any other R groups, as appropriate, given the number of Si atoms and available bonds in a given POSS molecule) are methyl, isobutyl or phenyl, etc.). This is particularly preferred for POSS molecules that have the structure of Formula I. Moreover, when a POSS molecule having 8 Si subunits, as depicted in Formula I, is employed, at least one of the R groups is a group other than a methyl.

Also contemplated under the term POSS is the family of commercially available compounds available from Hybrid Plastics, 18237 Mount Baldy Circle, Fountain Valley, Calif. 92708-6117 and Mayaterials, Inc. P.O. Box 87, South Lyon, Mich. 48178-0087.

Otherwise, POSS compounds with various R groups are well known in the literature. They are described in a number of U.S. patents including, for example, U.S. Pat. Nos. 5,047,492; 5,389,726; 5,484,867; 5,589,562; 5,750,741; 5,858,544; 5,939,576; 5,942,638; 6,100,417; 6,127,557; 6,207,364; 6,252,030; 6,270,561; 6,277,451; 6,362,279; and 6,486,254. These patents describe in detail various methods of producing the basic POSS cage structure and various derivatives thereof.

The amount of POSS-grafted polyolefin present in the cosmetic compositions of the present invention generally varies from about 1 to about 50%, and in some embodiments from about 5 to about 30% by weight, based on the total weight of the composition.

### Solvents

Suitable solvents (also “carriers”) for the POSS-grafted polyolefins are most typically non-aqueous or anhydrous in nature. The solvent should also be non-reactive with and in the presence of the POSS-grafted polyolefin as well as be cosmetically acceptable for purposes of use in a cosmetic or personal care product. Otherwise, they may be polar or non-polar, volatile or non-volatile, or aqueous or non-aqueous in nature.

Representative volatile solvents include non-polar volatile hydrocarbon-based oils (which as used herein, refers to oil containing only hydrogen and carbon atoms), silicone oils (optionally comprising alkyl or alkoxy groups that are pendant or at the end of a silicone chain), and fluoro oils. Suitable hydrocarbon-based oils include isoparaffins, e.g., branched alkanes containing 8-16 carbon atoms, such as isododecane (also known as 2,2,4,4,6-pentamethylheptane), and petroleum distillates. Suitable volatile silicone oils may include linear or cyclic silicones containing from 2 to 7 silicon atoms, these silicones optionally comprising alkyl or alkoxy groups containing from 1 to 10 carbon atoms. Examples include octamethylcyclotetrasiloxane, decamethylcyclopentasiloxane, hexadecamethylcyclohexasiloxane, heptamethylhexyltrisiloxane, decamethyltetrasiloxane and heptamethyloctyltrisiloxane.

Representative polar volatile solvents may also be used, examples of which include C₂ to C₅ alcohols, such as ethanol, ethyl 3-ethoxypropionate and isohexyl neopentanoate.

Representative non-polar non-volatile solvents include polyalphaolefins, which include ethylene derivatives oligomerized into even-numbered carbon polyalphaolefins e.g., C₆-C₁₄ olefins such as polydecene and polymers of C₆, C₈, C₁₂ and C₁₄ olefins. The polyolefins have a molecular weight (MW) generally ranging from about 280 to about 11,500, and a viscosity (CPs at about 20° C.) generally ranging from about 7 to about 32,500. They may also be hydrogenated. In some embodiments, the non-volatile polyolefin solvent may be obtained commercially from Exxon Chemicals under the tradename PureSyn™, e.g., PureSyn™ 2 (MW about 283), PureSyn™ 4 (MW about 432), PureSyn™ 6 (MW about 570), PureSyn™ 8 (MW about 611), PureSyn™ 150 (MW about 3980) and PureSyn™ 300 (MW about 4870) (INCI name: hydrogenated polydecene). The viscosities of these polymers are about 8, about 33, about 64, about 103, about 4179 and about 8400, respectively. PureSyn™ 100 (MW about 2939, viscosity about 3900, INCI name:hydrogenated C6-14 olefin polymers) and PureSyn™ 1000 (MW about 11,500, viscosity about 32,400, INCI name: polydecene) may also be useful.

Representative examples of non-volatile silicone oils which may be suitable for use as solvents/carriers include polydimethylsiloxanes (PDMSs), that are optionally phenylated, such as phenyltrimethicones, phenyltrimethylsiloxydiphenylsiloxanes, diphenylmethyldimethyltrisiloxanes, diphenyldimethicones, phenyldimethicones and polymethylphenylsiloxanes, optionally substituted with aliphatic and/or aromatic groups, or optionally fluorinated; polysiloxanes modified with fatty acids, fatty alcohols or polyoxyalkylenes (in particular polyoxyethylene or copoly(oxyethylene/oxypropylene) blocks or grafts such as dimethicone polyols); fluorosilicones and perfluorosilicone oils such as perfluoroalkyl polydimethylsiloxanes and perfluoroalkyl polymethylphenylsiloxanes; and silicones bearing both hydrophobic hydrocarbon-based groups (for example C₂-C₃₀ alkyl groups) and polyoxyethylenated or copoly(oxyethylenated/oxypropylenated) blocks or grafts, such as alkyldimethicone copolyols. Other non-volatile silicone oils that may be useful as solvents/carriers in the inventive compositions include dimethicone polymers available from Dow Corning under the name Dow Corning 200® Fluid and have viscosities ranging from 5 to 600,000 centistokes, the Viscasil series of polyalkylsiloxanes (General Electric Company) and the Dow Corning 200 series (Dow Corning Corp.), the polymethylphenyl siloxanes having viscosities of from about 15 to about 65 centistokes at 25° C. such as, for example, those available as SF 1075 methyl-phenyl fluid (General Electric Company) and 556 Cosmetic Grade Fluid (Dow Corning Corp.), polyethersiloxane copolymers such as a polyoxyalkylene ether copolymer having a viscosity of about 1200 to 1500 centistokes at 25° C., including for example, SF1066 organosilicone surfactant (General Electric Company).

The amount of solvent present in the cosmetic compositions of the present invention generally varies from about 10% to about 90%, and in some embodiments from about 20 to about 80% by weight, based on the total weight of the composition.

In view of the solubility of the POSS-grafted polyolefins, cosmetic compositions in which the POSS-grafted polyolefins may be formulated typically fall into two general categories, namely anhydrous-based compositions, and multiphasic compositions or emulsions, that include two or more phases, typically aqueous and oil-based, wherein the discrete (e.g., continuous and discontinuous) phases are dispersible by the presence of an emulsifier or other cosmetic ingredient with emulsifying properties.

Anhydrous compositions are typically characterized in that aside from an amount of water present in a pre-made commercial cosmetic ingredient, there is typically no added water. For purposes of the present invention, added water may be present in amounts of no more than 10%, 5%, 2% or even 1%, based on the total weight of the composition. Representative examples of anhydrous cosmetic compositions include non-compressed and compressed powders (such as foundation, and sticks), pastes, water-proof mascara, lipstick and lipgloss. Examples of non-anhydrous compositions include gels, lotions, solutions, foams and creams.

In addition to the POSS-grafted polyolefin and non-aqueous solvent, these compositions typically contain at least one additional cosmetic ingredient, including for example, structuring agents such as waxes and non-wax polymers, hydrophobic and hydrophilic gelling agents, and powders/fillers.

Emulsions typically contain, in addition to the POSS-grafted polyolefin and the solvent, at least one other phase e.g., a fatty or oil phase (that typically contains a liquid fatty phase and/or a fatty substance that is at least partially solid at room temperature (20° C.-25° C.)), or water, and an emulsifier or other cosmetic ingredient with emulsifying properties.

### Structuring Agents

The function of this ingredient is to structure (that is, thicken and/or increase the viscosity of) the product, and particularly an oil phase thereof, in order to form a solid product. Structuring agents that may be useful in the present invention include polyorganosiloxane-containing polymers, non-silicone-polyamide copolymers, waxes, and mixtures thereof. Polyorganosiloxane-containing polymers can generally be described as polymers chosen from homopolymers and copolymers, preferably, with a weight-average molecular mass ranging from about 500 to about 2.5×10⁶ or more, comprising at least one moiety comprising: at least one polyorganosiloxane group comprising, preferably, from 1 to about 10,000 organosiloxane units in the chain of the moiety or in the form of a graft, and at least two groups capable of establishing hydrogen interactions are provided. Non-silicone-polyamide copolymers include those known in the trade as Uniclear or Sylvaclear. These non-silicone polyamides have different terminal end groups, such as ester terminated, known as Uniclear 80 or 100, such as amide terminated, known as Sylvaclear A200, and such as polyalkyleneoxy terminated, known as Sylvaclear AF1900 as well as ester terminated polyesteramides. Such non silicone polyamides are commercially available, for instance, from Arizona Chemical Company, Jacksonville, Fla.

Suitable waxes are those generally used in cosmetics and dermatology. Representative examples of waxes include those of natural animal, plant or mineral origin, for instance beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methyl-siloxane esters that are solid at 40° C., for example, at above 55° C. Waxes approved for food use include ozokerite, rice wax and the waxes referenced in the Codex alimentary.

In general, the amount of structuring agent ranges from about 0.1 to about 30% and in some embodiments from about 0.5 to about 10% by weight, based on the total weight of the composition.

### Gelling Agents

These ingredients also referred to as gellants, thickeners or thickening agents, may be hydrophobic (and if water is present, hydrophilic) in nature. Representative examples of oil- or fatty-phase-compatible thickeners that may be suitable for use in the present invention may be polymeric or mineral-based. The thickener may cause gelling via chemical reticulation and agents that gel via physical reticulation. Modified clays may be used as thickeners, including hectorites modified with an ammonium chloride of a C10 to C22 fatty acid, such as hectorite modified with distearyldimethylammonium chloride, also known as quaternium-18 bentonite, such as the products commercially available from Rheox under the tradename Bentone 34, or from Southern Clay under the tradenames Claytone XL, Claytone 34 and Claytone 40, the modified clays known as quaternium-18 benzalkonium bentonites and commercially available from Southern Clay under the tradenames Claytone HT, Claytone GR and Claytone PS, the clays modified with stearyldimethylbenzoylammonium chloride, known as stearalkonium bentonites, such as those commercially available from Southern Clay under the tradenames Claytone APA and Claytone AF, and from Rheox under the tradename Baragel 24. Other mineral thickeners include silica, such as fumed silica.

Representative examples of hydrophilic or aqueous-compatible thickeners that may be useful in the present invention include polysaccharides and gums, e.g., natural gums, xanthan gum, sclerotium, carrageenan and pectin; polysaccharide resins such as starch and its derivatives, for example tapioca starch, polyvinylpyrrolidone (PVP), polyvinyl alcohol, crosslinked polyacrylic acids and acrylates (e.g., Carbopol 982), hydrophobically-modified acrylates (e.g., Carbopol 1382); polyacrylamides such as, for example, the crosslinked copolymers sold under the names Sepigel 305 (CTFA name: polyacrylamide/C13-C14 isoparaffin/Laureth 7) and Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; hyaluronic acid and its salts, clays such as montmorillonites, hectorites, bentonites, and laponites, polyglyceryl (meth)acrylates polymers commercially available from Hispano Quimica or Guardian under the tradenames “Hispagel” and “Lubragel”, crosslinked acrylamide polymers and copolymers, such as those commercially available from Hoechst under the tradenames “PAS 5161” and “Bozepol C”, and crosslinked methacryloyloxyethyltrimethylammonium chloride homopolymers such as those commercially available from Allied Colloid under the tradename “Salcare S.C.95”.

The gelling agent or thickener is typically present in an amount ranging from about 0.01% to about 10% by weight, in some embodiments from about 0.1% to about 5% by weight, based on the total weight of the composition.

### Powders/Fillers

These ingredients may be obtained from various sources (e.g., mineral or organic), and have any number of shapes (e.g., lamellar or spherical). Representative examples of fillers/powders that may be useful in the present invention include polyamide (Nylon) particles and especially the microbeads sold under the tradename Orgasol by the company Atochem, or nylon fibres; polyethylene powders; microspheres based on acrylic copolymers, such as those made of ethylene glycol dimethacrylate/lauryl methacrylate copolymer sold by the company Dow Corning under the tradename Polytrap; the polymethyl methacrylate microspheres sold under the tradename Microsphere M-100 by the company Matsumoto or under the tradename Covabead LH 85 by the company Wackherr; melamine-formaldehyde or urea-formaldehyde resin particles; poly(tetrafluoroethylene) particles; ethylene-acrylate copolymer powders, for instance those sold under the tradename Flobeads by the company Sumitomo Seika Chemicals; expanded powders such as hollow microspheres and especially microspheres formed from a terpolymer of vinylidene chloride, acrylonitrile and methacrylate, and sold under the tradename Expancel by the company Kemanord Plast under the references 551 DE 12 (particle size of about 12 μm and mass of a unit volume of 40 kg/m³), 551 DE 20 (particle size of about 30 μm and mass of a unit volume of 65 kg/m³) and 551 DE 50 (particle size of about 40 μm), or the polyacrylonitrile microspheres sold under the tradename Micropearl F 80 ED by the company Matsumoto; powders of natural organic materials such as starch powders, especially of crosslinked or non-crosslinked maize, wheat or rice starch, such as the powders of starch crosslinked with octenylsuccinate anhydride, sold under the tradename Dry-Flo by the company National Starch, and cellulose microbeads; and silicone resin microbeads, such as those sold under the tradename Tospearl by the company Toshiba Silicone, especially Tospearl 240.

The amount of filler/powder generally ranges from about 0.1% to about 25% and in some embodiments from about 1% to about 20% by weight, based on the total weight of the composition.

### Fatty Phase Ingredients

In addition to the non-aqueous solvent, at least one cosmetically or dermatologically acceptable and, in general, physiologically acceptable oil may be present. As used herein, the term “oil” means any fatty substance that is in liquid form at room temperature and atmospheric pressure. Oils that may be suitable for use in the present invention include both volatile and nonvolatile oils.

The volatile or nonvolatile oils are typically selected from hydrocarbon-based oils, silicone oils, and fluoro oils. The term “hydrocarbon-based oil” refers to oil mainly containing hydrogen and carbon atoms and possibly oxygen, nitrogen, sulfur and/or phosphorus atoms.

Representative categories of non-volatile hydrocarbon-based oils include fatty acids, linear or branched hydrocarbons of plant, mineral, or plant origin, and synthetic oils such as esters and ethers, fatty alcohols and fatty amides.

Examples of fatty acids include caprylic acid, capric acid, lauric acid, myristic acid, palmitic acid, stearic acid, oleic acid, ricinoleic acid, linoleic acid, linolenic acid, arachidic acid, gadoleic acid, behenic acid, erucic acid, brassidic acid, cetoleic acid, lignoceric acid and nervonic acid.

Examples of linear or branched hydrocarbons of mineral origin include mineral oils (e.g., paraffin), petroleum jelly, polydecenes, hydrogenated polyisobutene such as Parleam, perhydrosqualene and squalane.

Examples of hydrocarbon-based plant oils include triglycerides consisting of fatty acid esters of glycerol, the fatty acids of which may have chain lengths ranging from C4 to C24, these chains possibly being linear or branched, and saturated or unsaturated, e.g., heptanoic or octanoic triglycerides, groundnut oil, babassu oil, coconut oil, grapeseed oil, cottonseed oil, corn oil, corn germ oil, mustard seed oil, palm oil, rapeseed oil, sesame seed oil, soybean oil, sunflower oil, wheatgerm oil, canola oil, apricot oil, mango oil, castor oil, shea oil, avocado oil, olive oil, sweet almond oil, peach kernel oil, walnut oil, hazelnut oil, macadamia oil, jojoba oil, alfalfa oil, poppy seed oil, pumpkin oil, marrow oil, blackcurrant seed oil, evening primrose oil, millet oil, barley oil, quinoa oil, rye oil, safflower oil, candlenut oil, passionflower oil, musk rose oil or shea butter oil and alternatively caprylic/capric acid triglycerides.

Representative examples of synthetic esters and ethers, in particular of fatty acids, such as oils of formulae R1COOR2 and R1 OR2 in which R1 represents the residue of a fatty acid or of a fatty alcohol comprising from 8 to 29 carbon atoms and R2 represents a branched or unbranched hydrocarbon chain comprising from 3 to 30 carbon atoms, such as, for example, purcellin oil, octyl palmitate, isopropyl lanolate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate; or lipophilic derivatives of amino acids, such as isopropyl lauroyl sarcosinate (INCI name). Yet other examples include C₁₂-C₁₅ alkyl benzoates such as those sold under the tradenames “Finsolv TN” and “Witconol TN” by the company Witco, and 2-ethylphenyl benzoate, for instance the product sold under the name X-TEND 226® by the company ISP, triglycerides such as dicaprylyl carbonate (e.g., Cetiol CC, sold by Cognis), and oxyethylenated or oxypropylenated fatty esters and ethers.

Fatty alcohols which may be useful in the present invention tend to be liquid at room temperature and have a branched and/or unsaturated carbon-based chain containing from 12 to 26 carbon atoms. Representative examples thus include 2-octyldodecanol, isostearyl alcohol, oleyl alcohol, 2-hexyldecanol, 2-butyloctanol and 2-undecylpentadecanol.

Representative examples of fatty amides include isopropyl lauroyl sarcosinate such as the product sold under the tradename “Eldew SL-205” by the company Ajinomoto).

Representative examples of volatile hydrocarbon-based oils include oils containing from 8 to 16 carbon atoms, and especially branched C8-C16 alkanes (also known as isoparaffins), for instance isododecane (also known as 2,2,4,4,6-pentamethylheptane), isodecane and isohexadecane.

Examples of nonvolatile silicone oils that may be useful in the present invention include nonvolatile polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates, and dimethicones or phenyltrimethicones with a viscosity of less than or equal to 100 cSt.

Representative examples of volatile silicone oils that may be useful in the present invention include volatile linear or cyclic silicone oils, especially those with a viscosity ≦8 centistokes (8×10⁻⁶ m²/s) and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of volatile fluoro oils that may be suitable for use in the present invention include nonafluoromethoxybutane and perfluoro-methylcyclopentane.

The amount of oil that may present in the compositions generally ranges from about 5% to about 99% and in some embodiments, from about 10% to about 80% by weight, based on the total weight of the composition.

The fatty phase may contain any other standard fat-soluble or fat/oil-dispersible additive such as waxes and other polymeric structuring agents, and pasty compounds or substances, which as used herein, refer to fatty compounds with a reversible solid/liquid change of state and containing, at a temperature of 25° C., a liquid fraction and a solid fraction. Examples of pasty compounds, such as polyol esters, are described in U.S. Patent Application Publication 2010/0015074 A1.

The amount of fatty phase (including both liquids and solids), exclusive of emulsifier and hydrophobic gelling agent, that may present in the compositions generally ranges from about 5% to about 80% and in some embodiments, from about 10% to about 50% by weight, based on the total weight of the composition.

### Emulsifiers

Representative examples of emulsifiers that may be particularly suitable for use in the present invention include non-ionic amphiphilic lipids and anionic amphiphilic lipids.

Nonionic Amphiphilic Lipids:

The nonionic amphiphilic lipids of the invention are preferably chosen from 1) silicone surfactants; 2) amphiphilic lipids that are fluid at a temperature of less than or equal to 45° C., chosen from the esters of at least one polyol chosen from the group formed by polyethylene glycol comprising from 1 to 60 ethylene oxide units, sorbitan, glycerol comprising from 2 to 30 ethylene oxide units, polyglycerols comprising from 2 to 15 glycerol units, and of at least one fatty acid comprising at least one saturated or unsaturated, linear or branched C₈-C₂₂ alkyl chain; 3) mixed esters of fatty acid or of fatty alcohol, of carboxylic acid and of glycerol; 4) fatty acid esters of sugars and fatty alcohol ethers of sugars; 5) surfactants that are solid at a temperature of less than or equal to 45° C., chosen from fatty esters of glycerol, fatty esters of sorbitan and oxyethylenated fatty esters of sorbitan, ethoxylated fatty ethers and ethoxylated fatty esters; and 6) block copolymers of ethylene oxide (A) and of propylene oxide (B).

The silicone surfactants which can be used according to the invention are silicone compounds comprising at least one oxyethylene chain —OCH₂CH₂— and/or oxypropylene chain —OCH₂CH₂CH₂—. As silicone surfactants which can be used according to the present invention, mention may be made of those disclosed in documents U.S. Pat. No. 5,364,633 and U.S. Pat. No. 5,411,744.

The silicone surfactant used according to the present invention is preferably a compound of formula (VI):

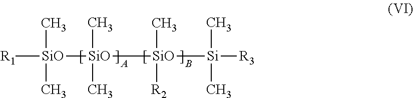

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical; A is an integer ranging from 0 to 200; B is an integer ranging from 0 to 50; with the proviso that A and B are not simultaneously equal to zero; x is an integer ranging from 1 to 6; y is an integer ranging from 1 to 30; and z is an integer ranging from 0 to 5.

According to one preferred embodiment of the invention, in the compound of formula (VI), the alkyl radical is a methyl radical, x is an integer ranging from 2 to 6 and y is an integer ranging from 4 to 30.

As examples of silicone surfactants of formula (VI), mention may be made of the compounds of formula (VII):

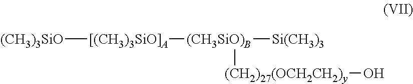

in which A is an integer ranging from 20 to 105, B is an integer ranging from 2 to 10 and y is an integer ranging from 10 to 20.

As examples of silicone surfactants of formula (VI), mention may also be made of the compounds of formula (VIII):

H—(OCH₂CH₂)_{y}—(CH₂)₃—[(CH₃)₂SiO]_{A′}—CH₂)₃—(OCH₂CH₂)_{y}—OH (VIII)

in which A′ and y are integers ranging from 10 to 20.

Compounds of the invention which may be used are those sold by the company Dow Corning under the names DC 5329, DC 7439-146, DC2-5695 and Q4-3667. The compounds DC 5329, DC 7439-146 and DC2-5695 are compounds of formula (II) in which, respectively, A is 22, B is 2 and y is 12; A is 103, B is 10 and y is 12; A is 27, B is 3 and y is 12.

The compound Q4-3667 is a compound of formula (VIII) in which A is 15 and y is 13.

The amphiphilic lipids that are fluid at a temperature of less than or equal to 45° C. are, in particular: the isostearate of polyethylene glycol of molecular weight 400, sold under the name PEG 400 by the company Unichema; diglyceryl isostearate, sold by the company Solvay; glyceryl laurate comprising 2 glycerol units, sold by the company Solvay; sorbitan oleate, sold under the name Span 80 by the company ICI; sorbitan isostearate, sold under the name Nikkol SI 10R by the company Nikko; and α-butylglucoside cocoate or α-butylglucoside caprate, sold by the company Ulice.

The mixed esters of fatty acid or of fatty alcohol, of carboxylic acid and of glycerol, which can be used as surfactants in the cosmetic composition according to the invention, may be chosen in particular from the group comprising mixed esters of fatty acid or of fatty alcohol with an alkyl chain containing from 8 to 22 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters which can be used in the cosmetic composition of the invention may be linear or branched, and saturated or unsaturated. They may especially be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

As examples of mixed esters which can be used in the cosmetic composition of the invention, mention may be made of the mixed ester of glycerol and of the mixture of citric acid, lactic acid, linoleic acid and oleic acid (CTFA name: Glyceryl citrate/lactate/linoleate/oleate) sold by the company Hills under the name Imwitor 375; the mixed ester of succinic acid and of isostearyl alcohol with glycerol (CTFA name: Isostearyl diglyceryl succinate) sold by the company Hills under the name Imwitor 780 K; the mixed ester of citric acid and of stearic acid with glycerol (CTFA name: Glyceryl stearate citrate) sold by the company Hills under the name Imwitor 370; the mixed ester of lactic acid and of stearic acid with glycerol (CTFA name: Glyceryl stearate lactate) sold by the company Danisco under the name Lactodan B30 or Rylo LA30.

Fatty acid esters of sugars, which can be used as surfactants in the cosmetic composition according to the invention, are preferably solid at a temperature of less than or equal to 45° C. and may be chosen in particular from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose.

The C₈-C₂₂ or C₁₄-C₂₂ fatty acids forming the fatty unit of the esters which can be used in the cosmetic composition of the invention comprise a saturated or unsaturated linear alkyl chain containing, respectively, from 8 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

As examples of esters or mixtures of esters of fatty acid and of sucrose, of maltose, of glucose or of fructose, mention may be made of sucrose monostearate, sucrose distearate and sucrose tristearate and mixtures thereof, such as the products sold by the company Croda under the name Crodesta F50, F70, F110 and F160 having, respectively, an HLB (hydrophilic lipophilic balance) of 5, 7, 11 and 16; and examples of esters or mixtures of esters of fatty acid and of methylglucose which may be mentioned are methylglucose polyglyceryl-3 distearate, sold by the company Goldschmidt under the name Tego-care 450. Mention may also be made of glucose or maltose monoesters such as methyl o-hexadecanoyl-6-D-glucoside and o-hexadecanoyl-6-D-maltoside.

The fatty alcohol ethers of sugars, which can be used as surfactants in the cosmetic composition according to the invention, are solid at a temperature of less than or equal to 45° C. and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty unit of the ethers which may be used in the cosmetic composition of the invention comprise a saturated or unsaturated, linear alkyl chain containing, respectively, from 8 to 22 or from 14 to 22 carbon atoms. The fatty unit of the ethers may be chosen in particular from decyl, cetyl, behenyl, arachidyl, stearyl, palmityl, myristyl, lauryl, capryl and hexadecanoyl units, and mixtures thereof, such as cetearyl.

As examples of fatty alcohol ethers of sugars, mention may be made of alkylpolyglucosides such as decylglucoside and laurylglucoside, which is sold, for example, by the company Henkel under the respective names Plantaren 2000 and Plantaren 1200, cetostearyl glucoside optionally as a mixture with cetostearyl alcohol, sold for example, under the name Montanov 68 by the company SEPPIC, under the name Tego-care CG90 by the company Goldschmidt and under the name Emulgade KE3302 by the company Henkel, as well as arachidyl glucoside, for example in the form of a mixture of arachidyl alcohol and behenyl alcohol and arachidyl glucoside, sold under the name Montanov 202 by the company SEPPIC.

The surfactant used more particularly is sucrose monostearate, sucrose distearate or sucrose tristearate and mixtures thereof, methylglucose polyglyceryl-3 distearate and alkylpolyglucosides.

The fatty esters of glycerol which may be used as surfactants in the cosmetic composition according to the invention, which are solid at a temperature of less than or equal to 45° C., may be chosen in particular from the group comprising esters formed from at least one acid comprising a saturated linear alkyl chain containing from 16 to 22 carbon atoms and from 1 to 10 glycerol units. One or more of these fatty esters of glycerol may be used in the cosmetic composition of the invention.

These esters may be chosen in particular from stearates, behenates, arachidates and palmitates, and mixtures thereof. Stearates and palmitates are preferably used.

As examples of surfactants which can be used in the cosmetic composition of the invention, mention may be made of decaglyceryl monostearate, distearate, tristearate and pentastearate (CTFA names: Polyglyceryl-10 stearate, Polyglyceryl-10 distearate, Polyglyceryl-10 tristearate, Polyglyceryl-10 pentastearate), such as the products sold under the respective names Nikkol Decaglyn 1-S, 2-S, 3-S and 5-S by the company Nikko, and diglyceryl monostearate (CTFA name: Polyglyceryl-2 stearate), such as the product sold by the company Nikko under the name Nikkol DGMS.

The fatty esters of sorbitan which may be used as surfactants in the cosmetic composition according to the invention are solid at a temperature of less than or equal to 45° C. and are chosen from the group comprising C₁₆-C₂₂ fatty acid esters of sorbitan and oxyethylenated C₁₆-C₂₂ fatty acid esters of sorbitan. They are formed from at least one fatty acid comprising at least one saturated linear alkyl chain containing, respectively, from 16 to 22 carbon atoms, and from sorbitol or from ethoxylated sorbitol. The oxyethylenated esters generally comprise from 1 to 100 ethylene glycol units and preferably from 2 to 40 ethylene oxide (EO) units.

These esters may be chosen in particular from stearates, behenates, arachidates, palmitates, and mixtures thereof. Stearates and palmitates are preferably used.

As examples of surfactants which can be used in the cosmetic composition of the invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The ethoxylated fatty ethers that are solid at a temperature of less than or equal to 45° C., which may be used as surfactants in the cosmetic composition according to the invention, are preferably ethers formed from 1 to 100 ethylene oxide units and from at least one fatty alcohol chain containing from 16 to 22 carbon atoms. The fatty chain of the ethers may be chosen in particular from behenyl, arachidyl, stearyl and cetyl units, and mixtures thereof, such as cetearyl. Examples of ethoxylated fatty ethers which may be mentioned are behenyl alcohol ethers comprising 5, 10, 20 and 30 ethylene oxide units (CTFA names: beheneth-5, beheneth-10, beheneth-20, beheneth-30), such as the products sold under the names Nikkol BB5, BB10, BB20 and BB30 by the company Nikko, and stearyl alcohol ether comprising 2 ethylene oxide units (CTFA name: steareth-2), such as the product sold under the name Brij 72 by the company ICI.

The ethoxylated fatty esters that are solid at a temperature of less than or equal to 45° C., which may be used as surfactants in the cosmetic composition according to the invention, are esters formed from 1 to 100 ethylene oxide units and from at least one fatty acid chain containing from 16 to 22 carbon atoms. The fatty chain in the esters may be chosen in particular from stearate, behenate, arachidate and palmitate units, and mixtures thereof. Examples of ethoxylated fatty esters which may be mentioned are the ester of stearic acid comprising 40 ethylene oxide units, such as the product sold under the name Myrj 52 (CTFA name: PEG-40 stearate) by the company ICI, as well as the ester of behenic acid comprising 8 ethylene oxide units (CTFA name: PEG-8 behenate), such as the product sold under the name Compritol HD5 ATO by the company Gattefosse.

The block copolymers of ethylene oxide (A) and of propylene oxide (B), which may be used as surfactants in the cosmetic composition according to the invention, may be chosen in particular from block copolymers of formula (IX):

HO(C₂H₄O)x(C₃H₆O)y(C₂H₄O)zH (IX)

in which x, y and z are integers such that x+z ranges from 2 to 100 and y ranges from 14 to 60, and mixtures thereof, and more particularly from the block copolymers of formula (I) having an HLB value ranging from 2 to 16.

These block copolymers may be chosen in particular from poloxamers and in particular Poloxamer 231, such as the product sold by the company ICI under the name Pluronic L81 of formula (XI) in which x=z=6, y=39 (HLB 2); Poloxamer 282, such as the product sold by the company ICI under the name Pluronic L92 of formula (XI) in which x=z=10, y=47 (HLB 6); and Poloxamer 124, such as the product sold by the company ICI under the name Pluronic L44 of formula (XI) in which x=z=11, y=21 (HLB 16).

Among the nonionic amphiphilic lipids that are preferably used are polyethylene glycol isostearate (8 mol of ethylene oxide), diglyceryl isostearate, polyglyceryl monolaurate and monostearate comprising 10 glycerol units, sorbitan oleate, and sorbitan isostearate.

Anionic Amphiphilic Lipids:

The anionic amphiphilic lipids of the invention are chosen in particular from Alkyl ether citrates, Alkoxylated alkenyl succinates, Alkoxylated glucose alkenyl succinates, and Alkoxylated methylglucose alkenyl succinates.

The alkyl ether citrates which may be used as surfactants in the cosmetic composition according to the invention may be chosen in particular from the group comprising monoesters, diesters or triesters formed from citric acid and from at least one oxyethylenated fatty alcohol comprising a linear or branched, saturated or unsaturated alkyl chain containing from 8 to 22 carbon atoms, and comprising from 3 to 9 ethoxylated groups, and mixtures thereof. Specifically, it is possible to use a mixture of one or more of these citrates in the cosmetic composition of the invention.

These citrates may be chosen, for example, from the mono-, di- and triesters of citric acid and of ethoxylated lauryl alcohol, comprising from 3 to 9 ethoxylated groups, which are sold by the company Witco under the name Witconol EC, in particular Witconol EC 2129 which is predominantly a dilaureth-9 citrate, and Witconol EC 3129 which is predominantly a trilaureth-9 citrate.

The alkyl ether citrates used as surfactants are preferably used in a form neutralized to a pH of about 7, the neutralizing agent being chosen from inorganic bases such as sodium hydroxide, potassium hydroxide or ammonia, and organic bases such as monoethanolamine, diethanolamine, triethanolamine, 1,3-aminomethylpropanediol, N-methylglucamine, basic amino acids such as arginine and lysine, and mixtures thereof.

The alkenyl succinates which may be used as surfactants in the cosmetic composition of the invention are, in particular, ethoxylated and/or propoxylated derivatives and they are preferably chosen from the compounds of formula (X) or (XI):

HOOC—(HR)C—CH₂—COO-E (X)

HOOC—(HR)C—CH₂—COO-E-O—CO—CH₂—C(HR′)—COOH (XI)

in which the radicals R and R′ are chosen from linear or branched alkenyl radicals containing from 6 to 22 carbon atoms, and E is chosen from oxyethylene chains of formula (C₂H₄O)ₙ in which n ranges from 2 to 100, oxypropylene chains of formula (C₃H₆O)ₙ in which n′ ranges from 2 to 100, random or block copolymers comprising oxyethylene chains of formula (C₂H₄O)ₙ and oxypropylene chains of formula (C₃H₆O)_{n′} such that the sum of n and n′ ranges from 2 to 100, the oxyethylenated and/or oxypropylenated glucose groups comprising on average from 4 to 100 oxyethylene and/or oxypropylene units distributed on all the hydroxyl functions, the oxyethylenated and/or oxypropylenated methylglucose groups comprising on average from 4 to 100 oxyethylene and/or oxypropylene units distributed on all the hydroxyl functions.

In formulae (X) and (XI), n and n′ are average values and are thus not necessarily integers. A value of n ranging from 5 to 60 and even more preferably from 10 to 30 is advantageously chosen.

The radical R and/or R′ is advantageously chosen from linear alkenyl radicals containing from 8 to 22 and preferably from 14 to 22 carbon atoms. It may be, for example, the hexadecenyl radical containing 16 carbon atoms or the octadecenyl radical containing 18 carbon atoms.

The compounds of formulae (X) and (XI) described above, in which E is chosen from oxyethylene chains, oxypropylene chains and copolymers comprising oxyethylene chains and oxypropylene chains, may be prepared in accordance with the description given in documents WO-A-94/00508, EP-A-107199 and GB-A-2 131 820, which are incorporated herein for reference.

The acid function —COOH in the surfactants of formulae (I) and (II) is generally in the cosmetic composition of the invention in a form which is neutralized with a neutralizing agent, the neutralizing agents being chosen, for example, from inorganic bases such as sodium hydroxide, potassium hydroxide or ammonia, and organic bases such as monoethanolamine, diethanolamine, triethanolamine, 1,3-aminomethylpropanediol, N-methylglucamine, basic amino acids such as arginine and lysine, and mixtures thereof.

As examples of surfactants which can be used in the cosmetic composition of the invention, mention may be made of hexadecenyl succinate 18 EO (compound of formula X with R=hexadecenyl, E=(C₂H₄O)ₙ, n=18), hexadecenyl succinate 45 EO (compound of formula X with R=hexadecenyl, E=(C₂H₄O)ₙ, n=45), dihexadecenyl succinate 18 EO (compound of formula XI with R=R′=hexadecenyl, E=(C₂H₄O)ₙ, n=18), dihexadecenyl glucose succinate 10 EO (compound of formula XI with R=R′=hexadecenyl, E=oxyethylenated glucose containing 10 oxyethylene groups), dihexadecenyl glucose succinate 20 EO (compound of formula XI with R=R′=hexadecenyl, E=oxyethylenated glucose containing 20 oxyethylene groups), dioctadecenyl methylglucose succinate 20 EO (compound of formula II with R=R′=octadecenyl, E=oxyethylenated methylglucose containing 20 oxyethylene groups), and mixtures thereof.

Depending on its more hydrophilic or more lipophilic nature, the nonionic or anionic amphiphilic lipid may be introduced into the aqueous phase or into the oily phase of the cosmetic composition.

Other Emulsifiers:

Cationic and amphoteric emulsifiers may also be useful.

The amount of emulsifier generally ranges from about 0.01 to about 20% and in some embodiments from about 0.1 to about 10% by weight, based on the total weight of the composition.

### Aqueous Phase

In addition to water, an aqueous phase may also include water-miscible or at least partially water-miscible compounds, such as polyols or lower C2 to C8 monoalcohols, such as ethanol and isopropanol. “Polyol” should be understood as meaning any organic molecule comprising at least two free hydroxyl groups, examples of which include glycols, such as butylene glycol, propylene glycol, isoprene glycol, glycerol and polyethylene glycols, such as PEG-8, sorbitol and sugars, such as glucose. The aqueous phase may further include any other water-soluble, cosmetically acceptable ingredient.

The amount of aqueous phase (water, water-miscible solvents and other aqueous components) generally ranges from about 0.1 to about 50% and in some embodiments from about 1 to about 30% by weight, based on the total weight of the composition.

The cosmetic compositions of the present invention may also contain at least one further cosmetically acceptable ingredient, which to the extent they are not already mentioned in connection with any specific category of composition, are typically selected from colorants, photoprotective agents (e.g., U.V. filters), secondary film-formers, fillers, cosmetically active agents, and/or cosmetic additives. These ingredients are selected based on several factors, including for example, their compatibility with the POSS-grafted polyolefin and the solvent system, and the intended overall effect of the composition.

Representative examples of all the forementioned cosmetic ingredients are provided below.

### Additional Film-Forming Polymers

These ingredients may be present in the inventive compositions, specifically selected depending on their compatibility with the POSS-polyolefin and the solvent. Broadly, film-forming polymers include synthetic polymers (of the free-radical type or the poly-condensate type), and polymers of natural origin. Of the term “free-radical film-forming polymer,” it is meant a polymer obtained by polymerization of unsaturated, e.g., ethylenically unsaturated monomers, capable of homopolymerization. Representative examples of these polymers that may be suitable for use in the present invention include vinyl polymers or copolymers, e.g., acrylic polymers. Vinyl film-forming polymers result from the polymerization of ethylenically unsaturated monomers containing at least one acidic group (e.g., α, β-ethylenic unsaturated carboxylic acids such as acrylic acid, methacrylic acid, crotonic acid, maleic acid, and itaconic acid), esters of the acid monomers (e.g., (meth)acrylates, such as (meth)acrylates of an alkyl, such as a C1-C30 and preferably C1-C20 alkyl, (meth)acrylates of an aryl, such as a C6-C10 aryl, and (meth)acrylates of a hydroxyalkyl, such as a C2-C6 hydroxyalkyl) and amides of the acid monomers (e.g., (meth) acrylamides, including N-alkyl (meth)acrylamides, such as a C2-C12 alkyl such as N-alkyl(meth)acrylamides, N-ethylacrylamide, N-t-butylacrylamide, N-t-octylacrylamide and N-undecylacrylamide). Vinyl film-forming polymers may also result from the homopolymerization or copolymerization of monomers selected from vinyl esters and styrene monomers, or copolymerization wherein these monomers are polymerized with the aforementioned acid, ester or amide monomers.

Representative examples of film-forming polycondensates that may be useful in the present invention include polyurethanes, polyesters, polyesteramides, polyamides, epoxy ester resins and polyureas.

The polymers of natural origin, which may be optionally modified, typically include shellac resin, sandarac gum, dammar resins, elemi gums, copal resins and cellulosic polymers.

Representative examples of specific oil/lipid-soluble film-forming polymers which may be suitable for use in the present invention include polyalkylenes, e.g., polybutene; alkylcelluloses with a linear or branched, saturated or unsaturated C1-C8 alkyl radical, e.g., ethylcellulose and propylcellulose; copolymers of vinylpyrrolidone (VP), e.g., copolymers of VP and C3-C20 alkenes, e.g., VP/vinyl acetate, VP/ethyl methacrylate, VP/eicosene, VP/hexadecene, and VP/styrene. Yet other oil/lipid-soluble film-forming polymers that may be useful include silicone resins, such as cross-linked polyorganosiloxanes and silicone resin copolymers. Block copolymers may also be useful (e.g., film-forming linear block ethylenic polymers which contain at least a first block and at least a second block with different glass transition temperatures that are linked together via an intermediate block containing at least one constituent monomer of the first block and at least one constituent monomer of the second block).

The film-forming polymer may also be present in an inventive composition in the form of particles dispersed in an aqueous phase (e.g., a (meth)acrylates copolymer) or in a non-aqueous solvent phase, which is generally known as a latex or pseudo latex.

Specific examples of representative polymers, including commercially available film-forming polymers are described in U.S. Patent Application Publication 2010/0278770 A1.

Additional film-forming polymers may be present in amounts generally ranging from about 0.1 to about 50% and in some embodiments from about 0.2 to about 40% by weight, based on the total weight of the composition.

### Colorants

Colorants may be chosen from the lipophilic dyes, hydrophilic dyes, traditional pigments, and nacres usually used in cosmetic or dermatological compositions, and mixtures thereof. The coloring agent may have any shape, such as, for example, spheroidal, oval, platelet, irregular, and mixtures thereof. Pigments may optionally be surface-treated e.g., with silicones (e.g., inorganic pigments may be coated with simethicone), perfluorinated compounds, lecithin, and amino acids.

The liposoluble dyes include, for example, Sudan Red, D&C Red 17, D&C Green 6, soybean oil, Sudan Brown, D&C Yellow 11, D&C Violet 2, D&C Orange 5, quinoline yellow and annatto. The water-soluble dyes are, for example, beetroot juice or methylene blue.

The pigments may be chosen from white pigments, colored pigments, inorganic pigments, organic pigments, coated pigments, uncoated pigments, pigments having a micron size and pigments not having a micron size. Among the inorganic pigments that may be mentioned are titanium dioxide, optionally surface-treated, zirconium oxide, zinc oxide, cerium oxide, chromium oxide, manganese violet, ultramarine blue, chromium hydrate, and ferric blue. Among the organic pigments which may be mentioned are carbon black, pigments of D&C type, lakes based on cochineal carmine, lakes based on barium, lakes based on strontium, lakes based on calcium, and lakes based on aluminum.

The nacreous pigments may, for example, be chosen from white nacreous pigments such as mica coated with titanium and mica coated with bismuth oxychloride, colored nacreous pigments such as titanium mica with iron oxides, titanium mica with, for example, ferric blue and/or chromium oxide, titanium mica with an organic pigment of the type mentioned above, as well as nacreous pigments based on bismuth oxychloride, interferential pigments, and goniochromatic pigments.

Colorants are generally present in an amount ranging from about 0.01% to about 20% and in some embodiments from about 0.1% to about 10%, by weight, based on the total weight of the composition.

### Photoprotectants

These ingredients which are also referred to as U.V. filters, can be organic or inorganic (or physical) agents. Representative examples of organic photoprotective agents that may be suitable for use in the present invention include dibenzoylmethane derivatives, e.g., butylmethoxydibenzoylmethane; cinnamic derivatives, e.g., ethylhexyl methoxycinnamate, isopropyl methoxycinnamate, isoamyl methoxycinnamate, DEA methoxycinnamate, diisopropyl methylcinnamate, and glyceryl ethylhexanoate dimethoxycinnamate; para-aminobenzoic acid derivatives, e.g., PABA, ethyl PABA, ethyl dihydroxypropyl PABA, ethylhexyl dimethyl PABA, glyceryl PABA, and PEG-25 PABA; salicylic derivatives, e.g., homosalate, ethylhexyl salicylate, dipropyleneglycol salicylate, and TEA salicylate; β,β-diphenylacrylate derivatives, e.g., octocrylene and etocrylene; benzophenone derivatives, e.g., benzophenone-1, benzophenone-2, benzophenone-3 (also known as oxybenzone), benzophenone-4, benzophenone-5, benzophenone-6, benzophenone-8, benzophenone-9, benzophenone-12, and n-hexyl 2-(4-diethylamino-2-hydroxybenzoyl) benzoate; benzylidenecamphor derivatives, e.g., 3-benzylidene camphor, 4-methylbenzylidene camphor, benzylidene camphor sulfonic acid, camphor benzalkonium methosulfate, terephthalylidene dicamphor sulfonic acid, and polyacrylamidomethyl benzylidene camphor; phenylbenzimidazole derivatives, e.g., phenylbenzimidazole sulfonic acid, and disodium phenyl dibenzimidazole tetrasulfonate; phenylbenzotriazole derivatives, e.g., drometrizole trisiloxane and methylene bis-benzotriazolyl tetramethylbutyl-phenol; triazine derivatives, e.g., bis-ethylhexyloxyphenol methoxyphenyl triazine, ethylhexyl triazone, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis(n-butyl 4′-aminobenzoate)-6-(aminopropyl-trisiloxane)-s-triazine, and 2,4-bis(dineopentyl 4′-aminobenzalmalonate)-6-(n-butyl 4′-aminobenzoate)-s-triazine; anthranilic derivatives, e.g., menthyl anthranilate; imidazoline derivatives, e.g., ethylhexyl dimethoxybenzylidene dioxoimidazoline propionate; benzalmalonate derivatives, e.g., polyorganosiloxane comprising benzalmalonate functional groups; 4,4-diarylbutadiene derivatives, e.g., 1,1-dicarboxy(2,2′-dimethylpropyl)-4,4-diphenylbutadiene; benzoxazole derivatives, e.g., 2,4-bis[5-1-(dimethylpropyl)benzoxazol-2-yl-(4-phenyl)imino]-6-(2-ethylhexyl)imino-1,3,5-triazine; and merocyanine derivatives, e.g., octyl 5-(N,N-diethylamino)-2-phenylsulfonyl-2,4-pentadienoate.

Preferred organic photoprotectants include octocrylene, homosalate, butylmethoxydibenzoylmethane, and ethylhexyl methoxycinnamate.

Representative inorganic photoprotectants are typically pigments formed of metal oxides which may or may not be coated (and which typically have a mean particle size between about 5×10⁻³ μm and 100×10⁻³ μm. Specific examples include pigments formed of titanium oxide, iron oxide, zinc oxide, zirconium oxide, and cerium oxide.

Representative examples of commercially available organic and inorganic photoprotective agents that may be useful in the present invention are disclosed, for example, U.S. Patent Application Publication 2010/0190740 A1.

Photoprotectants are generally present in an amount ranging from about 0.5 to about 50%, and in some embodiments from about 1 to about 40% by weight, based on the total weight of the composition.

Cosmetic Active Agents and other Additives

The compositions of the present invention may further contain at least one cosmetically active agent representative examples of which include anti-inflammatory agents, defoaming agents, emollients, vitamins, keratolytic and desquamating agents, α-hydroxy acids, depigmenting agents, salicylic acid, retinoids, hydrocortisone, natural extracts, steroids, anti-bacterial agents, enzymes, flavanoids, soothing agents, mattifying agents, trace elements and essential fatty acids. Aside from the forementioned fillers/powders, colorants, dispersion agents and photoprotectants, the compositions of the present invention may further contain at least one cosmetic additive representative examples of which include emollients, moisturizers, fibers, preservatives, chelators (such as EDTA and salts thereof, particularly sodium and potassium salts), antioxidants (e.g., BHT, tocopherol), essential oils, fragrances and neutralizing or pH-adjusting agents (e.g., sodium hydroxide). These ingredients may be selected for compatibility with aqueous or non-aqueous solvents (e.g., aqueous or fatty phase).

Cosmetic active agents and other cosmetic additives may present in the compositions in amounts generally ranging from about 0.01 to about 40% and in some embodiments from about 0.05 to about 30% by weight, based on the total weight of the composition.

The invention will now be discussed in terms of the following non-limiting examples. Unless otherwise specified, all parts are by weight.

### Examples 1 and 2

### Compositions Containing Colorant

| PHASE | TRADE NAME | EXAMPLE1 | EXAMPLE2 |
|---|---|---|---|
| A | PE-PP-POSS iBu | 11.52 | 0 |
| | 12 | | |
| | PE-PP-POSS Ph12 | 0 | 11.50 |
| | Isododecane | 73 | 73.01 |
| | Benton Gel | 4.42 | 4.42 |
| B | Color pigment | 3.98 | 3.98 |
| | Isododecane | 7.08 | 7.08 |
| | TOTAL | 100.00 | 100.00 |

The two polyolefin/POSS polymers used in both exemplified compositions are disclosed in Seurer et al., Macromol. Chem. Phys. 209:1198-1209 (2008). The cosmetic compositions described in the Table were made according to the following procedure: in phase A, the POSS containing polymer was dissolved in isododecane and the solution was stirred at 80-90° C.; after the solution became homogeneous, it was allowed to cool to room temperature; the bentone gel was added as in Phase A; and Phase B containing color pigment in isododecane, was added, with mixing for a couple of minutes until the composition became uniform. After drawing down the solution of examples 1 and 2, thus allowing the solvent to evaporate, the color films that formed were non-tacky, water-proof and showed good color transfer resistance.

All publications cited in the specification, both patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising a polymer having a chain comprising a polyolefin, and a polyhedral oligomeric silsesquioxane (POSS) grafted onto the polymer chain, a solvent and at least one other cosmetically acceptable ingredient.

2. The composition of claim 1, wherein monomeric POSS comprises a functional group reactive with a vinyl group on the polyolefin, and the POSS is grafted onto the polymer chain via the functional group.

3. The composition of claim 1, wherein monomeric POSS comprises a polymerizable functional group, and the polymer chain further comprises the polymerized functional group such that the POSS is grafted onto the polymer chain via the polymerized functional group.

4. The composition of claim 1, wherein the polyolefin is polyethylene, polypropylene, polyisobutene, polybutene, polyisoprene, polybutadiene, a polycycloalkene or a poly-hydrocarbon-diene.

5. The composition of claim 4, wherein the polycycloalkene comprises a polycyclooctene.

6. The composition of claim 4, wherein the poly-hydrocarbon diene comprises a pentylene-diene, or an olefinic copolymer thereof.

7. The composition of claim 1, wherein the polyolefin comprises a block copolymer.

8. The composition of claim 7, wherein the polyolefin block copolymer comprises polyethylene/polypropylene, styrene/butadiene/styrene, polyethylene/polypropylene/styrene or polycyclooctene/styrene.

9. The composition of claim 1, wherein the POSS is represented by formula I 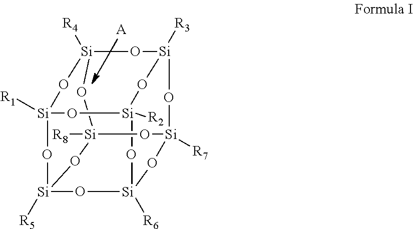
wherein R1-R8 each independently represents linear C1-10 groups, cyclic C3-12 alkyl groups, polymerizable functional groups and functional groups reactive with a vinyl group on the polyolefin, provided that at least one of R1-R8 is a polymerizable functional group or a functional group reactive with a vinyl group on the polyolefin.

10. The composition of claim 9, wherein the functional group reactive with a vinyl group on the polyolefin is a silane functional group or norbornene.

11. The composition of claim 9, wherein the polymerizable functional group is an ethylenically unsaturated group or an epoxy group.

12. The composition of claim 11, wherein the ethylenically unsaturated group is a substituted or unsubstituted acrylate, a substituted or unsubstituted methacrylate, a substituted or unsubstituted alkene, a substituted or unsubstituted acrylamide, norbornene, styrene, or a functional silicone.

13. The composition of claim 1, wherein the solvent comprises a volatile silicone oil a non-volatile silicone oil, or a combination thereof.

14. The composition of claim 1, which is anhydrous.

15. The composition of claim 1, further comprising water and an emulsifier.

16. The composition of claim 15, wherein the emulsifier comprises a nonionic amphiphilic lipid or an anionic amphiphilic lipid.

17. The composition of claim 1, wherein the at least one additional cosmetically ingredient comprises a structuring agent, a gelling agent, a powder, a filler, a colorant, and combinations of two or more thereof.

18. The composition of claim 17, wherein the structuring agent comprises a wax, a non-wax polymer, or a combination thereof.

19. A method for making up keratinous tissue, which comprises applying to the keratinous tissue the cosmetic composition of claim 1.

20. A method for making a cosmetic composition, comprising formulating a composition comprising a polymer having a chain comprising a polyolefin, and a POSSPOSS grafted onto the polymer chain, a solvent and at least one other cosmetically acceptable ingredient, into a cosmetic composition.

21. The composition of claim 1, which is in the form of a powder, paste, lipstick, lipgloss, a gel, a lotion, solution, foam, or a cream.
